(19) United States
(12) Patent Application Publication
(10) Pub. No.: US 2009/0068141 A1
(43) Pub. Date: Mar. 12, 2009

(54) AUTOLOGOUS NATURAL KILLER CELLS AND LYMPHODEPLETING CHEMOTHERAPY FOR THE TREATMENT OF CANCER

(75) Inventors: Maria R. Parkhurst, Ellicott City, MD (US); Steven A. Rosenberg, Potomac, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: The United States of America, as represented by the Secretary, Dept. of Health and Human Services, Rockville, MD (US)

(21) Appl. No.: 12/205,106

(22) Filed: Sep. 5, 2008

Related U.S. Application Data
(63) Continuation-in-part of application No. PCT/US2007/063352, filed on Mar. 6, 2007.
(60) Provisional application No. 60/779,863, filed on Mar. 6, 2006.

(51) Int. Cl. A61K 35/12; A61K 38/20; A61K 39/395; A61P 35/00; A61P 31/18

(52) U.S. Cl. 424/85.2; 424/93.71; 424/155.1

(57) ABSTRACT

The invention provides a simple, cost-effective method of preparing a composition comprising natural killer (NK) cells useful for administering to a human. The method comprises (i) depleting CD3⁺ cells from a population of PBMCs comprising NK cells, and (ii) co-culturing cells from (i) with irradiated PBMCs that are autologous to the NK cells. Further provided by the invention are the compositions prepared thereby and methods of treating or preventing a disease or immunodeficiency in a host.

### CROSS-REFERENCE TO RELATED APPLICATIONS

This patent application is a continuation-in-part of International Patent Application No. PCT/US2007/063352, filed Mar. 6, 2007, which claims the benefit of U.S. Provisional Patent Application No. 60/779,863, filed Mar. 6, 2006, both of which are incorporated by reference.

### BACKGROUND OF THE INVENTION

Previous and current clinical investigations have clearly demonstrated that T lymphocytes can mediate the regression of metastatic melanoma (Rosenberg and Dudley, Proc. Natl. Acad. Sci. U.S.A. 101 Suppl 2: 14639-14645 (2004)). In one such trial conducted in the Surgery Branch of the National Cancer Institute (Dudley et al., J. Clin. Oncol. 23: 2346-2357 (2005)), tumor reactive T lymphocyte populations were isolated from tumor infiltrating lymphocytes (TIL) and were expanded to large numbers (i.e., 10¹⁰ cells) ex vivo. These cells were then adoptively transferred to autologous patients with interleukin 2 (IL-2) after the patients had been treated with a lymphodepleting, but nonmyeloablative, regimen of chemotherapy (cyclophosphamide and fludarabine). Of the 35 patients treated in this investigation, 18 experienced objective clinical responses (51%).

However, not all patients with cancer are eligible for this type of immunotherapy. In some patients, the TIL do not expand sufficiently, or do not exhibit sufficient tumor specific reactivity. Also, the isolation and maintenance of tumor reactive cytotoxic T lymphocytes (CTL) from TIL or peripheral blood lymphocytes (PBL) stimulated in vitro with tumor cells has been largely unsuccessful for the treatment of breast, prostate, and colon cancers. Furthermore, as shown in the afore-mentioned clinical trial, the durations of the responses to TIL therapy can be short-lived, and recurrent tumors sometimes fail to express the class I MHC molecules typically needed for T lymphocyte recognition.

An alternative type of therapy involves the adoptive transfer of autologous natural killer (NK) cells. Studies in mice have shown that adoptive transfer of NK cells activated in vitro can significantly reduce the load of Acute Myelogenous Leukemia (AML) (Siegler et al., Leukemia 19: 2215-2222 (2005)), and intravenously-injected autologous NK cells have been shown to significantly decrease melanoma tumor outgrowths (Lozupone et al., Cancer Res. 64: 378-385 (2004)). Other studies demonstrate that adoptively transferred NK cells undergo homeostatic proliferation in a lymphopenic environment (Prlic et al., J. Exp. Med. 197: 967-976 (2003); Jamieson et al., J. Immunol. 172: 864-870 (2004)). Also, CD4⁺CD25⁺ regulatory T cells (Treg) were shown to inhibit NKG2D-mediated NK cell cytotoxicity in vitro, and depletion of Tregs in vivo significantly enhanced tumor rejection mediated by NK cells (Smyth et al., J Immunol. 176: 1582-1587 (2006)). However, because these studies involved adoptive transfer of human cells into mice, these studies are not necessarily predictive of the effects of adoptively transferring autologous NK cells to humans.

Adoptive transfer of a mixed population of cells comprising autologous NK cells for the treatment of humans with melanoma, renal cell carcinoma, lymphoma, and breast cancer has been addressed in several previously described clinical trials using ex vivo generated lymphokine activated killer (LAK) cells (Rosenberg et al., N. Engl. J. Med. 313:1485-1492 (1985); Burns et al., Bone Marrow Transplant. 32: 177-186 (2003)). However, a clear clinical benefit was not observed in these trials. Also, the efficacy of autologous NK cell adoptive transfer cannot be determined from these previous studies, since the studies involved the use of LAK cells, which consist predominantly of T lymphocytes (>90%) and contain only a small fraction (<10%) of cells having the phenotypic characteristics of classical NK cells (i.e., CD56⁺/CD3⁻).

In view of the foregoing, there remains a need for methods and compositions, especially autologous methods and compositions, useful for the treatment, prevention, and research of cancer.

### BRIEF SUMMARY OF THE INVENTION

An embodiment of the invention provides a method of preparing a composition comprising NK cells, which method comprises (i) depleting CD3⁺ cells from a population of PBMCs to provide a CD3⁺ cell-depleted population of PBMCs, wherein the population of PBMCs comprises NK cells, and (ii) co-culturing cells from the CD3⁺ cell-depleted population of PBMCs with irradiated PBMCs, wherein the irradiated PBMCs are autologous to the NK cells. The invention also provides an NK cell composition prepared by the above method.

The invention further provides a method of treating or preventing a disease, especially cancer, or an immunodeficiency, in a host. An embodiment of the method comprises administering to the host a composition comprising autologous NK cells in an amount effective to treat the disease or immunodeficiency, wherein the autologous NK cells are ex vivo-activated by co-culturing with irradiated autologous PBMCs.

An embodiment of the invention also provides a method of treating cancer in a host that has undergone lymphodepleting chemotherapy, which method comprises administering to the host a composition comprising ex vivo-activated autologous NK cells in an amount effective to treat the cancer.

### BRIEF DESCRIPTION OF THE SEVERAL VIEWS OF THE DRAWING(S)

FIGS. 1A-1I are flow cytometry graphs illustrating the phenotypic cell populations of PBMCs in whole PBMC fractions (FIGS. 1A, 1D, and 1G), in PBMC fractions after CD3⁺ cell depletion (FIGS. 1B, 1E, and 1H), and after co-culturing with irradiated PMBCs for 21-31 days (FIGS. 1C, 1F, and 1I).

FIG. 2 is a graph of the fold expansion of PBMCs as a function of time (days). The line with ♦ indicates Donor 1; ▪ indicates Donor 2; and ▴ indicates Donor 3.

FIGS. 3A-3L are flow cytometry graphs illustrating the phenotype of a population of NK cells grown under a large-scale expansion protocol. FIG. 3A shows the population of cells labeled with FITC-conjugated anti-CD56 and PE-conjugated anti-CD3, corresponding to the basic phenotype of CD56⁺ and CD3⁻. FIGS. 3B and 3C show the population of cells labeled with FITC- or PE-conjugated antibodies specific for CD56 or NK inhibitory receptors: CD158a and CD158b. FIGS. 3D-3H show the population of cells labeled with FITC- or PE-conjugated antibodies specific for CD56 or NK activating receptors: CD16, NKG2D, CD69, NKp46, and CD94. FIGS. 3I-3L show the population of cells labeled with FITC- or PE-conjugated antibodies specific for CD56 or cytokine receptors: CD127R (IL-7R), CD25R, and γ and β chains of the IL-2 receptor.

FIGS. 4A-4C are graphs of the degree of lysis of target melanoma cells (888 mel (□), A375 (▪), SK23 mel (∘), 624 mel ()) and control target cells (PBMCs (⋄)) observed at different effector cell:target cell (E:T) ratios.

FIGS. 5A-5C are graphs of the degree of lysis of target melanoma cells (888 melanoma (HLA⁺; ▪) and 1858 melanoma (HLA⁻; ▴)) and renal cell carcinoma cells (WA RCC () and WH RCC (♦) and control target cells (PBMCs (∘)) observed at different E:T ratios.

FIG. 6 is a flow chart of a method of a positive selection or depletion using CliniMACS® CD3 MicroBeads following an In-Bag-Preparation protocol.

### DETAILED DESCRIPTION OF THE INVENTION

An embodiment of the invention provides a method of preparing a composition comprising NK cells, which method comprises (i) depleting CD3⁺ cells from a population of PBMCs to provide a CD3⁺ cell-depleted population of PBMCs, wherein the population of PBMCs comprises NK cells, and (ii) co-culturing cells from the CD3⁺ cell-depleted population of PBMCs with irradiated PBMCs, wherein the irradiated PBMCs are autologous to the NK cells.

The population of PBMCs comprising NK cells referred to in (i) of the inventive method can be obtained through any suitable method known in the art. For example, the population of PBMCs comprising NK cells can be obtained by a leukapheresis of a blood sample taken from a host. Other methods of isolating or otherwise obtaining a suitable population of PBMCs comprising NK cells are known in the art.

The term “host” as used herein encompasses any host. Preferably, the host is a mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Camivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

The depletion of CD3⁺ cells from the population of PBMCs can be performed by any suitable method. Suitable methods of depleting CD3⁺ cells from a population of PBMCs are known in the art. For instance, the CD3⁺ cells can be depleted through fluorescent activated cell sorting (FACS) using an appropriately labeled anti-CD3 antibody, e.g., FITC-conjugated anti-CD3 or PE-conjugated anti-CD3 antibody, etc. Alternatively, the CD3⁺ cells can be depleted from the population of PBMCs though column chromatography, e.g., affinity chromatography using anti-CD3 antibodies. Also, the CD3⁺ cells can be depleted from a population of PBMCs through the use of a kit comprising a biotin-conjugated antibody against CD3, as well as beads labeled with anti-biotin antibodies. Such kits are commercially available. In a preferred embodiment of the inventive method, the CD3⁺ cells are depleted from the population of PBMCs by using a CliniMACS® System (Miltenyi Biotec) and CD3 reagent (Miltenyi Biotec).

Depletion of CD3⁺ cells from the population of PBMCs can be performed to any degree. Preferably, depletion of CD3⁺ cells is sufficient to remove about 50% or more, preferably about 75% or more, about 80% or more, about 90% or more, about 95% or more, or about 99% or more (e.g., substantially all or all) of the CD3⁺ cells from the population of PBMCs.

While the CD3⁺ cell-depleted PBMC population also can be depleted of other cell phenotypes (e.g., CD4⁺, CD14⁺, CD15⁺, CD19⁺, CD36⁺, CD 123 cells), desirably the CD3+ cell-depleted PBMC population is depleted of as few other cell phenotypes, other than CD3+ cells, as possible prior to co-culturing with the irradiated PBMCs. Thus, the CD3⁺ cell-depleted PBMC population is preferably not depleted of more than about three additional cell phenotypes, more preferably not more than about two or even one additional cell phenotype. Most desirably, the CD3⁺ cell-depleted PBMC population is not depleted of any cell phenotypes other than the CD3⁺ cells. This aspect of the method is advantageous in that it simplifies the method of preparing the composition, and it is believed to be beneficial in that the PBMC population is less significantly changed by removing only CD3⁺ cells as compared to removing more cell types.

The irradiated PBMCs can be provided by any suitable method. Any PBMC population can be irradiated to provide the irradiated PBMCs, provided that the PBMCs are autologous to the NK cells of the CD3⁺ depleted population of PBMCs. Suitable PBMCs can be obtained by any of the methods previously described herein with respect to the population of PBMCs used in (i) of the method, which comprises the NK cells. The PBMCs used for irradiation can, for example, be provided by a fraction of the same PBMCs used in (i) of the method, described above. Preferably, the irradiated PBMCs are obtained by leukapheresis of a blood sample of a host. More preferably, the irradiated PBMCs are from the same host as the PBMCs comprising the NK cells, used in (i) of the method. In this regard, a method of preparing an NK cell composition can comprise (i) depleting CD3⁺ cells from a first portion of a population of PBMCs, wherein the first portion of PBMCs comprises NK cells, to provide a CD3⁺ cell-depleted population of PBMCs, (ii) irradiating a second portion of the population of PBMCs to provide irradiated PBMCs, and (iii) co-culturing the CD3⁺ cell-depleted population of PBMCs with the irradiated PBMCs. The PBMCs can be irradiated by any suitable method. Methods of irradiating PBMCs are known in the art (e.g., Dudley et al., J. Clin. Oncol. 23: 2346-2357 (2005)) and described herein.

The irradiated PBMCs and CD3⁺ cell-depleted PBMCs can be co-cultured by any suitable method. Methods of culturing cells are known in the art (see, e.g., Tissue Engineering Methods and Protocols, Morgan and Yarmush (eds.), Humana Press, Inc., Totowa, N.J., 1999). Of course, the conditions under which cells are cultured varies depending on the cell type, e.g., cell phenotype. The conditions include temperature of the environment, the culturing vessel containing the cells, the composition of the various gases, e.g., CO₂, which comprises the cell culture atmosphere or environment, the medium in which the cells are maintained, the components and pH of the medium, the density at which cells are maintained, the schedule by which the medium needs to be replaced with new medium, etc. It is within the skill of the ordinary artisan to determine the optimum parameters for a given cell culture. In one embodiment, the cells are co-cultured in a medium comprising IL-2 and OKT3. Alternatively, the cells are co-cultured in a medium comprising interleukin-12 (IL-12). It is believed that co-culturing the cells in the presence of IL-12 stimulates NK cells to produce cytokines (e.g., interferons) that enhance the therapeutic effect of the NK cells (e.g., lysing target cancer cells). The medium also can contain other reagents including heat inactivated human AB serum. One method of co-culturing the cells is described in Example 1.

The cells can be co-cultured for any amount of time, such as about 1 day or more (e.g. about 1-3 days), about 4 days or more (e.g., about 4-7 days), about 1 week or more (e.g., about 8-13 days), about 2 weeks or more (e.g., about 2-3 weeks, or about 14-18 days, or about 19-21 days), about 3 weeks or more (e.g., about 21-25 days or about 26-31 days), or about 4 weeks or more (e.g., about 32 days or more). In a preferred embodiment of the inventive method, the cells are co-cultured for at least 21 days, at least 31 days, or about 21 to about 31 days (e.g., about 21 to about 28 days). In another preferred embodiment, the cells are co-cultured for 21 to 25 days.

Without wishing to be bound by any particular theory, it is believed that co-culturing the CD3⁺ cell-depleted PBMCs comprising NK cells with irradiated PBMCs that are autologous to the NK cells yields conditions which permit optimal proliferation (i.e., expansion) and activation of the NK cells, such that the NK cell composition prepared in accordance with the method of the invention comprises a significant population of activated NK cells.

Activated NK cells express at increased levels one or more of the NK activating receptors NKG2D, CD16, NKp46, and CD94. The NK cell composition prepared by an embodiment of the method of the invention preferably comprises a population of NK cells exhibiting an increased expression level of one or more of the NK activating receptors as compared to the NK cells of the population of PBMCs prior to CD3⁺ cell depletion and/or co-cultivation with irradiated PBMCs.

It is further preferred that the NK cells of the NK cell composition prepared by an embodiment of the method of the invention, in addition to or instead of expressing one or more NK activating receptors at increased levels, are able to effectively lyse target cells, e.g., virally-infected or tumor (cancer) cells. Preferably, the NK cells of the NK cell composition are able to lyse target cancer cells, such as the cells of any of the cancers described herein. In a more preferred embodiment, the NK cells of the prepared composition are able to lyse melanoma cells. Desirably, the NK cells of the NK cell composition prepared by the method of the invention can lyse target cells with equal or greater efficiency than the NK cells of the PBMCs prior to prior to CD3⁺ cell depletion and/or co-cultivation with irradiated PBMCs.

An embodiment of the method of preparing an NK cell composition provides for the significant expansion of NK cells in culture. Preferably, the number of NK cells of the prepared composition is at least about 25-fold greater, more preferably at least about 50-fold greater, or even at least about 100-fold greater or 1000-fold greater than the number of NK cells in the CD3⁺ cell-depleted PBMC population prior to co-culturing with the irradiated PBMCs.

The NK cell composition prepared in accordance with an embodiment of the invention can comprise a population of immune cells other than NK cells, but preferably comprises a significant portion of ex-vivo activated autologous NK cells. For instance, the prepared composition can comprise a population of immune cells in which at least about 25% or more of the population is ex vivo-activated autologous NK cells. Preferably, the composition comprises a population of immune cells in which at least about 50% of the population is ex vivo-activated autologous NK cells. More preferably, the composition comprises a population of immune cells in which at least about 75% of the population is ex vivo-activated autologous NK cells. Most preferably, the composition comprises a population of immune cells in which at least about 98% of the population is ex vivo-activated autologous NK cells. Desirably, the NK cell composition consists essentially of ex vivo-activated autologous NK cells, meaning that it is substantially free of cells (e.g., contains less than about 20%, 15%, 10%, 5%, 2%, or 1% of the total population of cells) that counteract the ability of the autologous NK cells to expand in culture, or inhibit the biological activity of the ex vivo-activated autologous NK cells.

The NK cells of the NK cell composition can be genetically modified. In this respect, the NK cells of the NK cell composition can comprise a heterologous nucleic acid sequence which encodes a protein. A “heterologous nucleic acid sequence” can be any nucleic acid sequence that is not obtained or derived from a naturally occurring nucleic acid sequence of an NK cell. Alternatively, a “heterologous nucleic acid sequence” can be naturally found an NK cell, but located at a normative position within genome of the NK cell and/or operably linked to a normative promoter. The NK cells can comprise at least one heterologous nucleic acid sequence, i.e., the NK cells can comprise one heterologous nucleic acid sequence or more than one heterologous nucleic acid sequence (i.e., two or more heterologous nucleic acid sequences). The heterologous nucleic acid sequence preferably encodes a protein (i.e., one or more nucleic acid sequences encoding one or more proteins). An ordinarily skilled artisan will appreciate that any type of nucleic acid sequence (e.g., DNA, RNA, and cDNA) that can be inserted into a cell can be used in connection with the invention.

In the context of the invention, the heterologous nucleic acid sequence encodes a cytokine or an Fc receptor. Cytokines are known in the art as non-antibody proteins secreted by specific cells (e.g., inflammatory leukocytes and some non-leukocytic cells), that act as intercellular mediators, such as by regulating immunity, inflammation, and hematopoiesis. Cytokines generally act locally in a paracrine or autocrine rather than endocrine manner. Cytokines can be classified as a lymphokine (cytokines made by lymphocytes), a monokine (cytokines made by monocytes), a chemokine (cytokines with chemotactic activities), and an interleukin (cytokines made by one leukocyte and acting on other leukocytes). The cytokine can be any suitable cytokine known in the art, including, but not limited to, interferons, interleukins, RANTES, MCP-1, MIP-1α, and MIP-11 granulocyte monocyte colony-stimulating factor (GM-CSF), and tumor necrosis factor (TNF) alpha. Preferably, the cytokine is interleukin-12 (IL-12). An Fc receptor is a protein found on the surface of certain immune system cells (e.g., NK cells, macrophages, neutrophils, and mast cells) which bind to antibodies that are attached to infected cells or invading pathogens. When activated, Fc receptors stimulate phagocytic or cytotoxic cells to destroy pathogens or infected cells by antibody-mediated phagocytosis or antibody-dependent cell-mediated cellular cytotoxicity (ADCC). The heterologous nucleic acid sequence can encode any suitable Fc receptor including, but not limited to, FcγRI (CD64), FcγRIIA (CD32), FcγRIIB (CD32), FcγRIIIA (CD16a), FcγRIIIB (CD16b), FcαRI (CD89), FcεRI, and FcεRII. Fc receptors are described in, e.g., Fridman, FASEB J., 5(12): 2684-2690 (1991), Trinchieri et al., Nat. Immun., 12: 218-234 (1993), and Janeway et al. (eds.), Immunobiology, 5^{th} ed., Garland Publishing, New York, N.Y. (2001). Methods for introducing heterologous nucleic acid sequences into cells (e.g., mammalian cells) are known in the art and described in, for example, Ausubel et al. (eds.), Short Protocols in Molecular Biology, 5^{th} ed., John Wiley & Sons, Inc., Hoboken, N.J. (2002).

Preferably, the heterologous nucleic acid is operably linked to (i.e., under the transcriptional control of) one or more promoter and/or enhancer elements, for example, as part of a promoter-variable expression cassette. Techniques for operably linking sequences together are well known in the art. A “promoter” is a DNA sequence that directs the binding of RNA polymerase and thereby promotes RNA synthesis. A nucleic acid sequence is “operably linked” to a promoter when the promoter is capable of directing transcription of that nucleic acid sequence. A promoter can be native or non-native to the nucleic acid sequence to which it is operably linked.

Methods of testing NK cells for biological activity, increased expression of NK activating receptors, and proliferation are known in the art. For example, a ⁵¹Cr release assay can be used to measure the lytic activity of NK cells, as described in Pinilla-Ibarz et al., Haematologica 90:1324-1332 (2005), Igarashi et al., Blood 104: 170-177 (2004), and in Example 1. Also, for example, expression levels of NK activating receptors can be assayed by quantitative Western blot (e.g., Western blot followed by phosphorimaging) or FACS analysis using antibodies specific for the NK activating receptors, which methods are described in Wang et al., Drug Metab. Disposition 32: 1209-1214 (2004); Igarashi et al., 2004, supra, and Example 1. Methods of measuring NK cell proliferation include thymidine incorporation assays and FACS analysis using antibodies specific for CD56 and CD3, which methods are described in Ogier et al., BMC Neurosci.6: 68-., Igarashi et al., 2004, supra, and Example 1 herein.

Compositions, such as, for example, pharmaceutical compositions, comprising NK cells prepared by the inventive method are further provided by the invention. The inventive compositions can comprise other components in addition to the NK cells. For example, the pharmaceutical composition can comprise NK cells in combination with other pharmaceutically active agents or drugs, such as one or more of chemotherapeutic agents (e.g., cyclophsphamide, fludaribine, asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.), cytokines (e.g., IL-2, IL-12, IL-15, and the like), or other agents (e.g., OKT3).

The compositions preferably comprise a carrier. Preferably, the carrier is a pharmaceutically acceptable carrier. With respect to pharmaceutical compositions, the carrier can be any of those conventionally used and is limited only by chemico-physical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular inventive composition, as well as by the particular method used to administer the inventive composition. Accordingly, there are a variety of suitable formulations of the pharmaceutical composition of the invention. The following formulations for parenteral, intravenous, intramuscular, intra-arterial, intrathecal, and intraperitoneal administration are exemplary and are in no way limiting. More than one route can be used to administer the inventive composition, and in certain instances, a particular route can provide a more immediate and more effective response than another route.

Injectable formulations are in accordance with the invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Toissel, 4th ed., pages 622-630 (1986)). Preferably, when administering cells, e.g., NK cells, the cells are administered via injection. The injection can be administered to the host in any manner, including but not limited to, intravenously, intraperitoneally, intramuscularly, intrathecally, or intra-arterially. Preferably, the injection is administered to the host intravenously.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The inventive compositions comprising NK cells can be administered in a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5% to about 25% by weight of the inventive composition in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

For purposes of the invention, the amount or dose of the inventive composition administered should be sufficient to effect, e.g., a therapeutic or prophylactic response, in the subject or animal over a reasonable time frame. For example, the dose of the composition should be sufficient to lyse target tumor or cancer cells in a period of about 2 hours or longer, e.g., 12 to 24 or more hours, from the time of administration. In certain embodiments, the time period could be even longer. The dose will be determined by the efficacy of the particular inventive composition and the condition of the animal (e.g., human), as well as the body weight of the animal (e.g., human) to be treated.

Many assays for determining an administered dose are known in the art. For purposes of the invention, an assay, which comprises comparing the extent to which target cells are lysed upon administration of a given dose of a composition to a mammal among a set of mammals of which is each given a different dose of the composition, could be used to determine a starting dose to be administered to a mammal. The extent to which target cells are lysed upon administration of a certain dose can be assayed by methods known in the art, including, for instance, the methods described herein as Example 1.

The dose of the inventive compositions also will be determined by the existence, nature and extent of any adverse side effects that might accompany the administration of a particular inventive composition. Typically, the attending physician will decide the dosage of the inventive composition with which to treat each individual patient, taking into consideration a variety of factors, such as age, body weight, general health, diet, sex, inventive composition to be administered, route of administration, and the severity of the condition being treated. By way of example and not intending to limit the invention, the dose of the inventive composition can be about 1.0×10¹⁰ NK cells to about 7.5×10¹⁰ NK cells, e.g., about 1.5×10¹⁰ NK cells, about 2.5×10¹⁰ NK cells, about 5.0×10¹⁰ NK cells, about 6.0×10¹⁰ NK cells, etc.

One of ordinary skill in the art will readily appreciate that the compositions of the invention can be modified in any number of ways, such that the therapeutic or prophylactic efficacy of the inventive compositions is increased through the modification.

As stated above, the inventive method allows for the substantial isolation, expansion, and activation of NK cells, which NK cells are particularly useful for administration to a host for purposes of treating or preventing a disease or an immunodeficiency in a host. In this regard, the invention provides a method of treating or preventing a disease or an immunodeficiency in a host. An embodiment of the method comprises administering to the host a composition comprising autologous NK cells in an amount effective to treat the disease or the immunodeficiency, wherein the autologous NK cells are ex vivo-activated by co-culturing with irradiated autologous PBMCs.

For purposes herein, “immunodeficiency” means the state of a host whose immune system has been compromised by disease or by administration of chemicals. This condition makes the system deficient in the number and type of blood cells needed to defend against a foreign substance. The immunodeficiency treated or prevented by the inventive method can be any immunodeficiency, such as, for example, Acquired Immunodeficiency Syndrome (AIDS), Severe Combined Immunodeficiency Disease (SCID), selective IgA deficiency, common variable immunodeficiency, X-linked agammaglobulinemia, chronic granulomatous disease, hyper-IgM syndrome, and diabetes. Preferably, the immunodeficiency is AIDS.

The disease treated or prevented by the inventive method can be an autoimmune disease. For purposes herein, “autoimmune disease” refers to a disease in which the body produces an immunogenic (i.e., immune system) response to some constituent of its own tissue. In other words the immune system loses its ability to recognize some tissue or system within the body as “self” and targets and attacks it as if it were foreign. Autoimmune diseases can be classified into those in which predominantly one organ is affected (e.g., hemolytic anemia and anti-immune thyroiditis), and those in which the autoimmune disease process is diffused through many tissues (e.g., systemic lupus erythematosus). For example, multiple sclerosis is thought to be caused by T cells attacking the sheaths that surround the nerve fibers of the brain and spinal cord. This results in loss of coordination, weakness, and blurred vision. Autoimmune diseases are known in the art and include, for instance, Hashimoto's thyroiditis, Grave's disease, lupus, multiple sclerosis, rheumatic arthritis, hemolytic anemia, anti-immune thyroiditis, systemic lupus erythematosus, celiac disease, Crohn's disease, colitis, diabetes, scleroderma, psoriasis, and the like. Preferably, the autoimmune disease is an autoimmune disease which directly or indirectly causes a depletion, dysfunction, or malfunction of NK cells in the diseased host.

Alternatively, the disease can be an infectious disease. For purposes herein, “infectious disease” means a disease that can be transmitted from person to person or from organism to organism, and is caused by a microbial agent (e.g., common cold). Infectious diseases are known in the art and include, for example, hepatitis, sexually transmitted diseases (e.g., Chlamydia, gonorrhea), tuberculosis, HIV/AIDS, diphtheria, hepatitis B, hepatitis C, cholera, and influenza. For purposes herein, the infectious disease preferably is one which is caused by or involves a viral infection.

Also, the disease to be treated or prevented by the inventive method can be a tumor or a cancer. With respect to the inventive method of treating or preventing a disease or immunodeficiency in a host, when the disease is cancer, the cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor. Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. Preferably, the cancer is melanoma, renal cell carcinoma, or breast, prostate, or colon cancer.

In this regard, the invention further provides a method of treating cancer in a host. The method comprises administering to the host a composition comprising autologous ex vivo-activated NK cells in an amount effective to treat the cancer.

With respect to the inventive methods, the host can be any host as previously described herein. Preferably, the host is a mammal, and, more preferably, the host is a human. In a preferred embodiment of the invention, the host is a host that has undergone lymphodepleting chemotherapy. More preferably, the lymphodepleting chemotherapy is a nonmyeloablative lymphodepleting chemotherapy, such as a regimen of cyclophosphamide and fludaribine. Without wishing to be bound by any particular theory, it is believed that the combination of the lymphodepleting chemotherapy and subsequent administration of the composition comprising autologous ex-vivo activated NK cells provides an enhanced therapeutic effect.

In another preferred embodiment, the host is a host that has undergone adoptive transfer of autologous tumor infiltrating lymphocytes (TIL), and/or the host is a host from which tumor-reactive T cells can not be generated or from which tumor-reactive T cells can not be ex vivo-activated. It is contemplated that such hosts are hosts for which the inventive method are particularly well-suited.

In view of the foregoing, the method of treating cancer can comprise any number of additional aspects. For example, the method can further comprise administering to the host a lymphodepleting chemotherapy before, during, or after the administration of the composition comprising autologous ex vivo-activated NK cells. Alternatively or additionally, the method of treating cancer can further comprise adoptive transfer of autologous tumor infiltrating lymphocytes (TIL) before, during, or after the administration of the composition comprising autologous ex vivo-activated NK cells. Also, the method can comprise, for example, administering IL-2 and/or IL-12 to the host before, during, or after administration of the composition comprising the autologous ex vivo activated NK cells. Preferably, the IL-2 and/or IL-12 is administered at the same time that the NK cells are administered to the host.

In another embodiment, the method can comprise administering antibodies, such as tumor-specific antibodies to the host before, during, or after administration of the composition comprising the autologous ex vivo activated NK cells. Preferably, the antibody is a monoclonal antibody. Monoclonal antibodies have been developed for the treatment of cancer. Any suitable tumor-specific monoclonal antibody can be used in connection with the invention. Suitable tumor-specific monoclonal antibodies include, but are not limited to, Rituximab (Rituxan®, Genentech, South San Francisco, Calif.), Trastuzumab (Herceptin®, Genentech, South San Francisco, Calif.), Gemtuzumab ozogamicin (Mylotarg®, Wyeth, Madison, N.J.), Alemtuzumab (Campath®, Genzyme Corporation, Cambridge, Mass.), Ibritumomab tiuxetan (Zevalin®, Cell Therapeutics, Inc., Seattle, Wash.), Tositumomab (Bexxar®, GlaxoSmithKline, London), Cetuximab (Erbitux®, ImClone Systems, Inc., New York, N.Y., and Bristol-Myers Squibb, Princeton, N.J.), Bevacizumab (Avastin®, Genentech, South San Francisco, Calif.), and Panitumumab (Vectibix®, Amgen, Inc., Thousand Oaks, Calif.). It is believed that coupling the adoptive transfer of NK cells with the administration of tumor-specific monoclonal antibodies enhances tumor cell death via antibody-dependent cell-mediated cellular cytotoxicity (ADCC).

With respect to the inventive method of treating cancer in a host, the cancer can be any cancer, including any of those described herein. In one embodiment, the cancer is historically responsive to IL-2 immunotherapy, e.g., melanoma. In another embodiment, the cancer is renal cell carcinoma or breast, prostate, or colon cancer.

In one embodiment of the invention, the cancer cells express do not express any Major Histocompatibility Complex (MHC) Class I molecules. For example, the cancer cells can be cancer cells which have lost expression of MHC Class I molecules. The cancer cells can alternatively or additionally lose expression of other MHC molecules, such as MHC Class II molecules or minor MHC molecules.

In another embodiment of the invention, the cancer cells express an MHC molecule, e.g., a Class I, Class II, or minor MHC molecule. However, the cancer cells can be cancer cells which express an MHC molecule to a lesser extent as compared to a corresponding non-cancerous cell. In this regard, the cells of the cancer can have a decreased expression of a MHC molecule. Preferably, the cells of the cancer have a decreased expression of an HLA-B or an HLA-C molecule, or a decreased expression of both HLA-B and HLA-C molecules.

With respect to any of the inventive methods of treating or preventing a disease, including the inventive method of treating cancer, the composition administered to the host can be any of the inventive compositions described herein (e.g., prepared by the method of preparing an NK cell composition as described herein). For instance, the composition can be a composition comprising ex vivo-activated autologous NK cells which are prepared by ex vivo co-culturing the NK cells with irradiated PBMCs that are autologous to the NK cells. Thus, the method of treating or preventing a disease can further comprise any one or more steps or aspects of the method of preparing a composition comprising NK cells, as described herein.

As used herein, the terms “treat,” and “prevent” as well as words stemming therefrom, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

The following examples further illustrate the invention but, of course, should not be construed as in any way limiting its scope.

### EXAMPLE 1

This example demonstrates a clinically-applicable method of preparing NK cells for adoptive transfer into cancer patients in accordance with one embodiment of the invention.

The PBMCs from each of three leukaphereses (two fresh leukaphereses and one cryopreserved leukapheresis) are subjected to the following ex vivo expansion protocol. A first portion of the leukapheresed PBMCs is depleted of CD3⁺ cells using a CliniMACSR System and CD3 reagent (Miltenyi Biotec, Auburn, Calif.). A second portion of the leukapheresed PBMCs are irradiated with 3000 rad using a ¹³⁷Cs irradiator, as described in Dudley et al., 2005, supra. Multiple T175 flasks are then set up, each of which contained 10⁷ CD3 depleted cells and 10⁸ irradiated autologous PBMCs as feeder cells in 100 ml AIMV media containing 10% heat inactivated human AB serum in the presence of 100 CU/ml IL-2 and 30 ng/ml OKT3. On the third to fourth day of co-culturing the depleted cells and irradiated cells, 100 CU/ml IL-2 is added, and, on day 7-8, fresh media containing 5% human AB serum (100 ml) are added to each flask. On or about day 10 of co-culturing, the contents of three flasks are transferred to a single 2-L LifeCell culture bag (Baxter, Deerfield, Ill.), and the cell concentration of the culture bag is adjusted to 0.5×10⁶ cells/ml with AIMV media containing 5% human AB serum containing 100 CU/ml IL-2. Cells are maintained as needed by adding fresh serum-free AIMV media and 100 CU/ml IL-2 and/or splitting the cultures to maintain a cell concentration between 1−3×10⁶ cells/ml. The cells are cultured in this manner for 21 to +31 days.

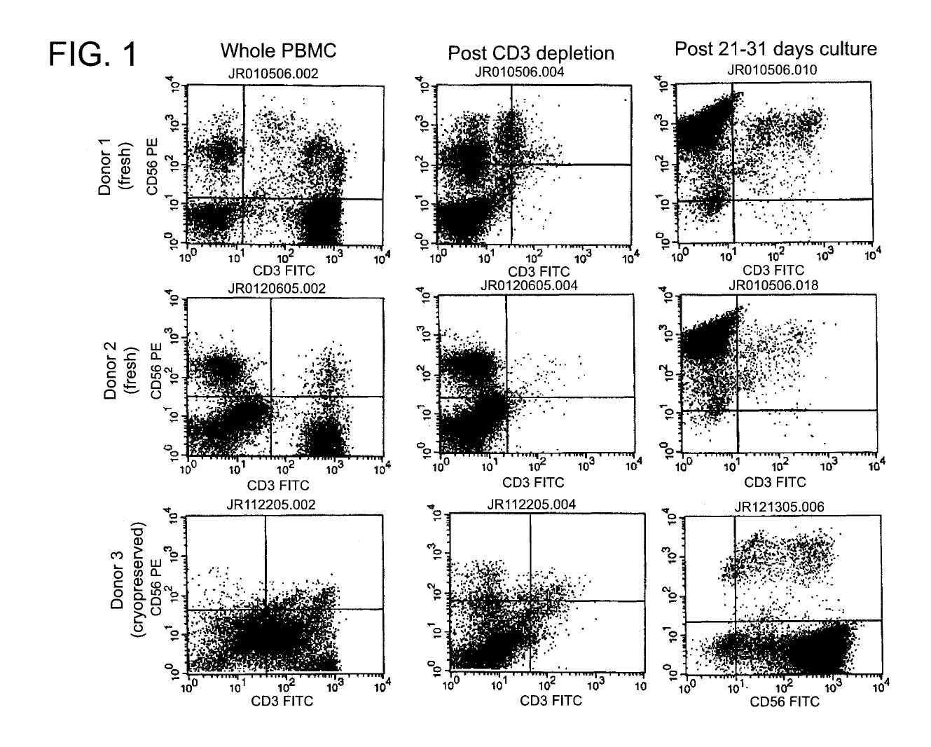

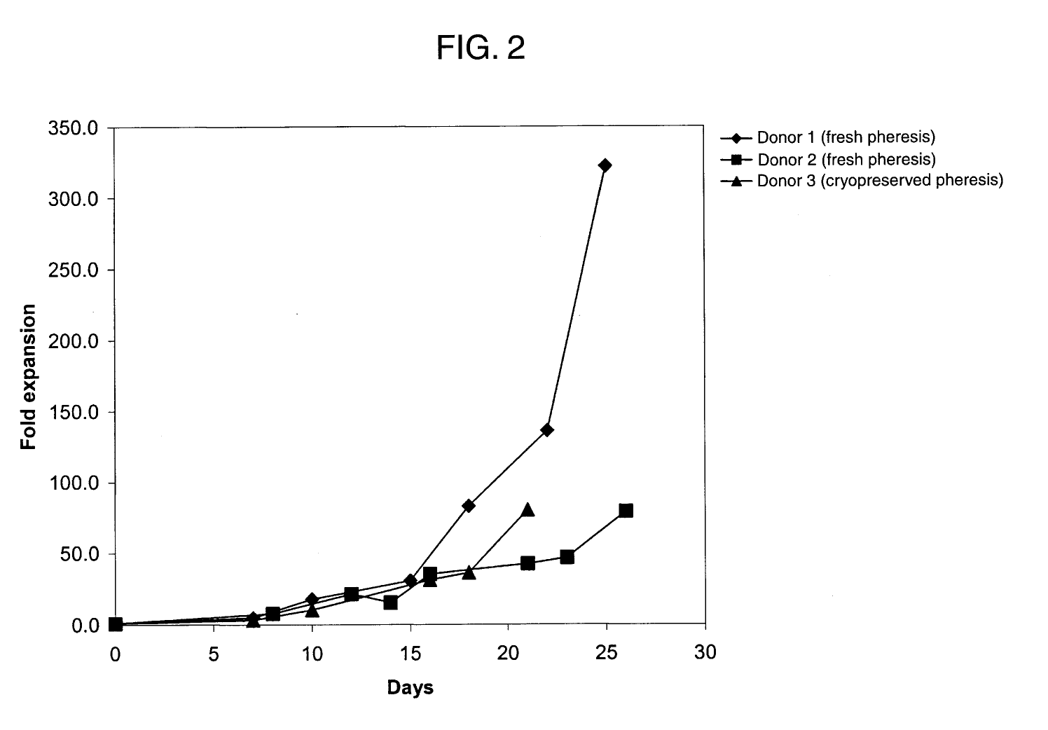

The NK proliferation of the ex vivo expanded cells is measured by staining an aliquot of the cultured cells (˜1×10⁶) with phycoerythrin (PE)-conjugated anti-CD56 antibodies (BD Pharmingen, San Jose, Calif.) and fluorescein-5-isothiocyanate (FITC)-conjugated anti-CD3 antibodies (BD Pharmingen) and analyzing by FACS analysis. As shown in FIG. 1, NK cell proliferation is dominant during the culture period of 21 to 31 days. As shown in FIG. 2, a minimum 50-fold expansion is achieved between days 21 and 25, regardless of whether the cells originated from a fresh or cryopreserved leukapheresis.

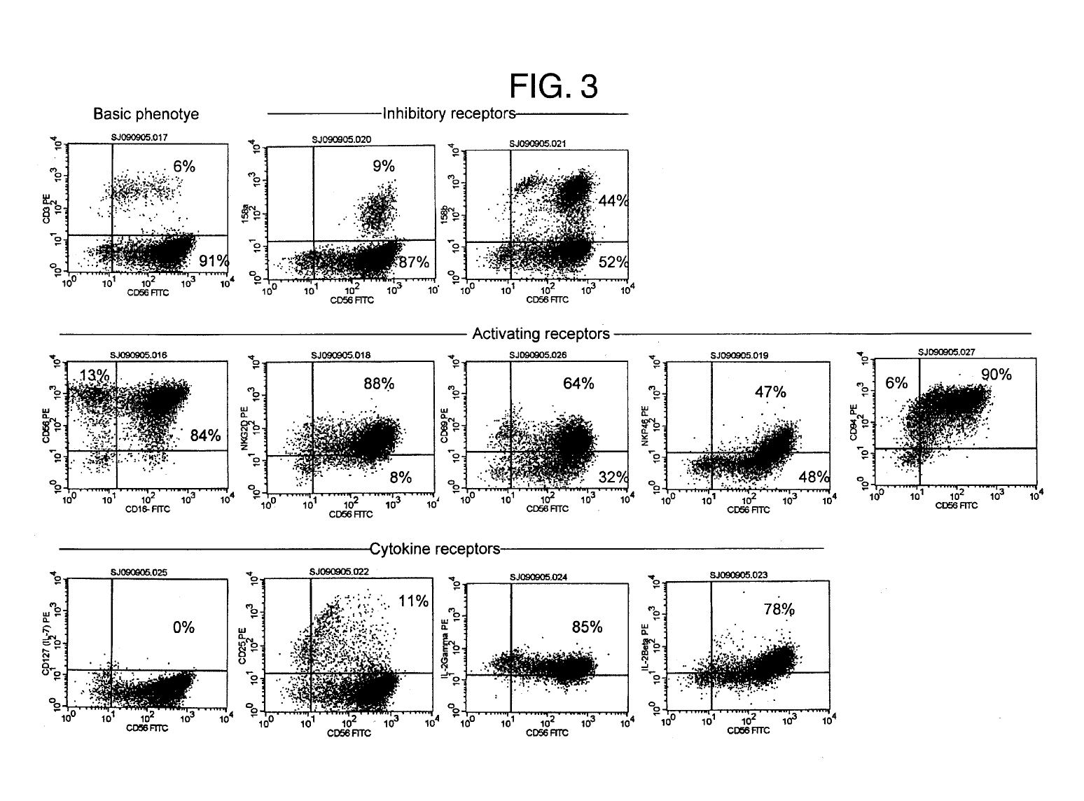

The phenotypes of the ex vivo expanded cells are also evaluated by FACS analyses by staining aliquots of cells (˜1×10⁶) with two of the following antibodies: PE-conjugated anti-CD56, PE-conjugated anti-CD3, PE-conjugated anti-CD127, PE-conjugated anti-CD25, PE-conjugated NKG2D, PE-conjugated anti-CD 158a, FITC-conjugated anti-CD158b, PE-conjugated anti-CD69, PE-conjugated anti-NKP46, PE-conjugated anti-CD94, PE-conjugated anti-IL-2γ chain, PE-conjugated anti-IL-20 chain, FITC-conjugated anti-CD16, and FITC-conjugated anti-CD56 (BD Pharmingen). The phenotypes of ex vivo expanded cells are similar in terms of expression of activating and inhibitory natural killer cells receptors (NKRs) to the phenotypes of cells of preliminary experiments in which NK cells are isolated using an NK cell isolation kit (Miltenyi Biotec) and expanded by co-culturing with irradiated allogeneic PBMCs. Namely, the cells appear to be highly activated NK cells with upregulated expression of activating NKRs: NKG2D, CD16, NKp46, and CD94 (FIG. 3).

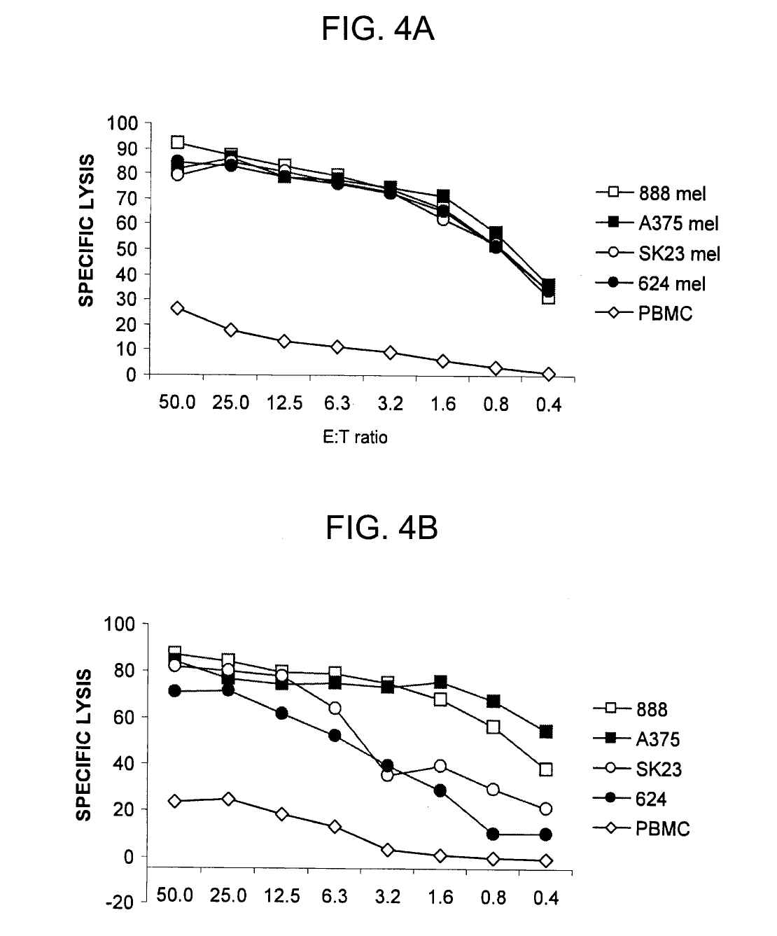

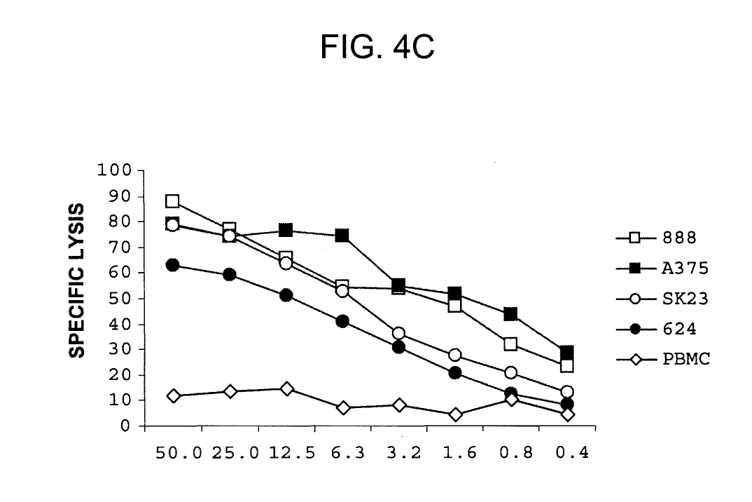

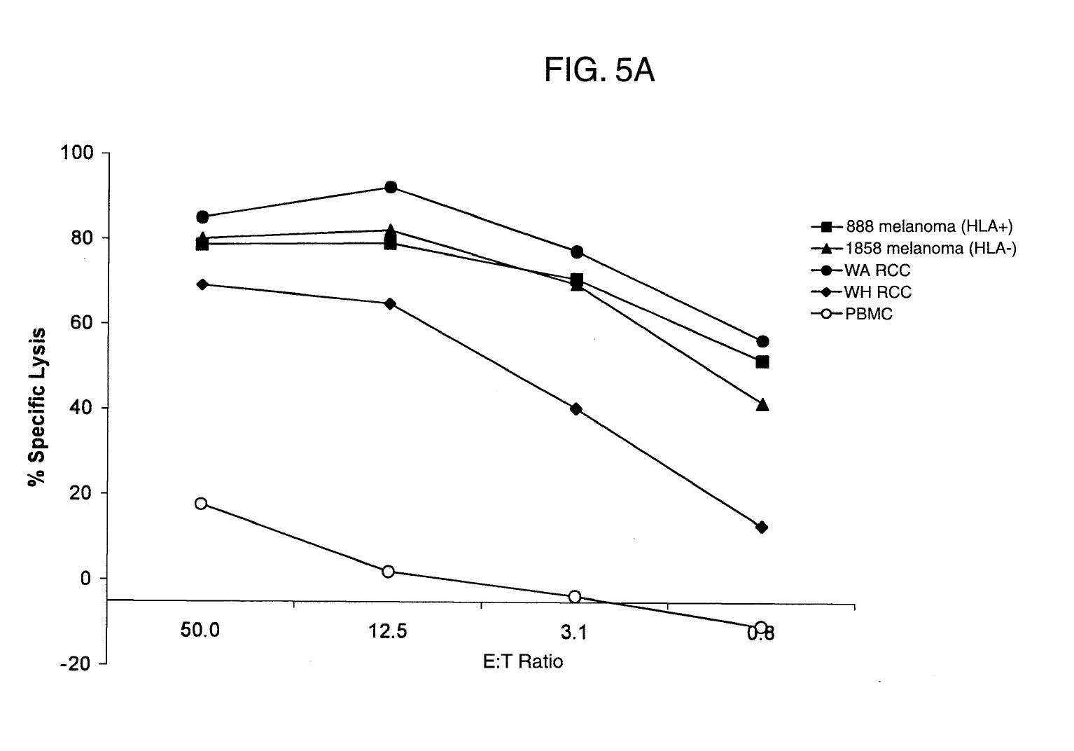

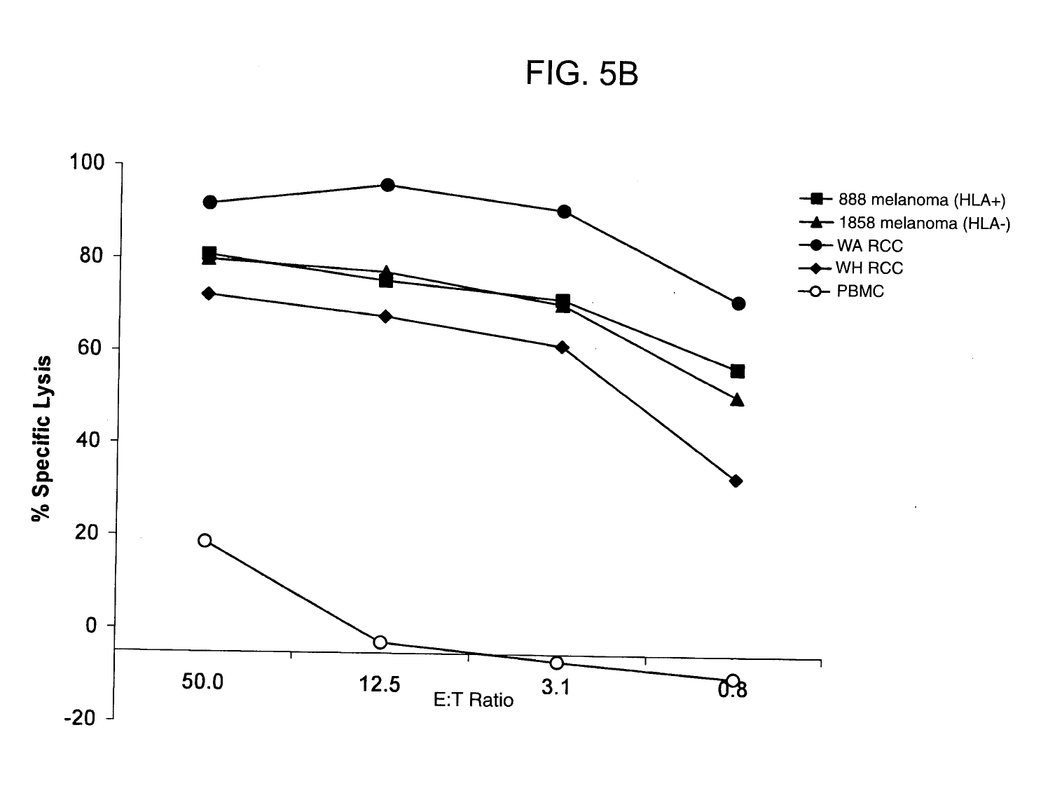

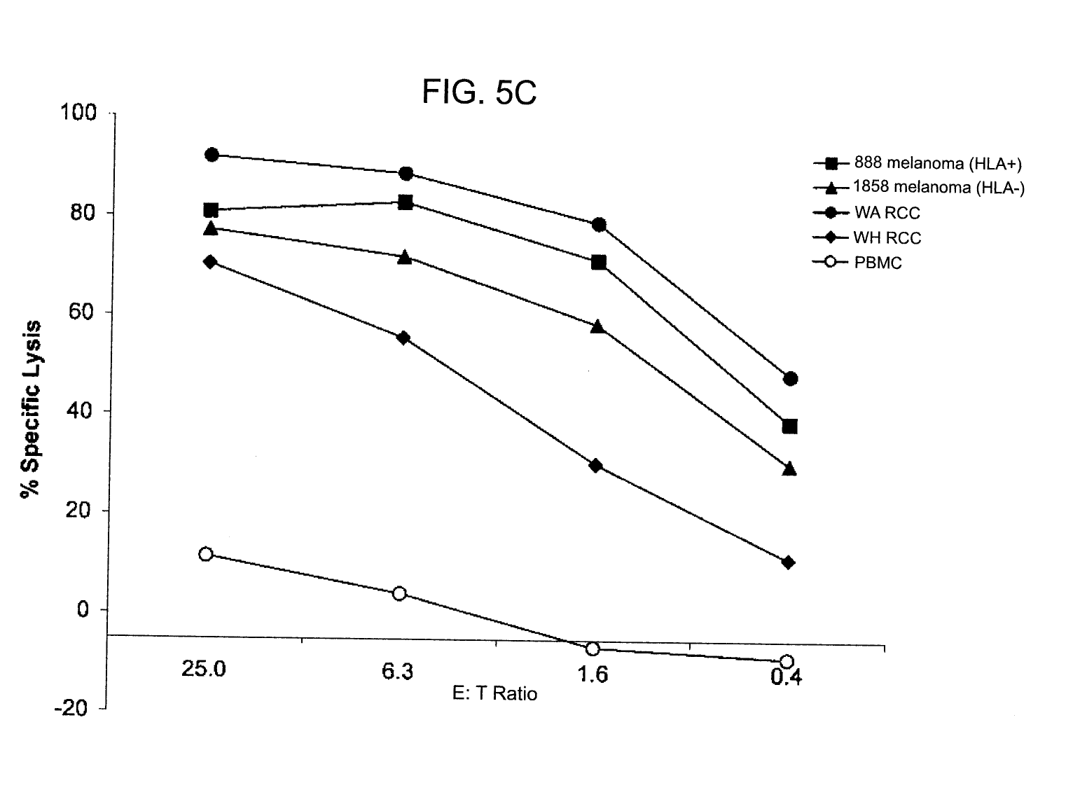

The lytic function of the ex vivo expanded NK cells is evaluated by measuring the release of ⁵¹Cr-labeled target cells, as described in (Igarashi et al., 2004, supra). Briefly, melanoma tumor cells: 888 mel, A375 mel, SK23 mel, and 624 mel, and negative control cells (PBMCs) are incubated with ⁵¹Cr for 1 hour. Ex vivo expanded NK cells (effector cells) are co-incubated with target cells at different effector to target (E:T) ratios. As shown in FIGS. 4 and 5, the ex vivo expanded NK cells from all three leukaphereses are capable of lysis of melanoma cells. The NK cells did not lyse PBMCs.

This example demonstrated a clinical method of preparing biologically active, autologous NK cells for adoptive transfer into diseased patients.

### EXAMPLE 2

This example demonstrates the adoptive transfer of autologous NK cells into a cancer patient that has undergone lymphodepleting chemotherapy for the treatment of cancer in accordance with one embodiment of the invention.

PBMCs (10¹⁰) from a leukapheresis of cancer Patient X are divided into two aliquots: one for CD3 depletion and the other reserved for irradiation. PBMCs are depleted for CD3 or are irradiated as described in Example 1. CD3 depleted cells (5×10⁹) and irradiated PBMCs (5×10⁹) are distributed into fifty T175 flasks, each flask containing equal amounts of CD3 depleted cells and irradiated PBMCS. The depleted cells and irradiated cells are then co-cultured as described in Example 1. The biological activity of the ex vivo expanded cells are tested as described in Example 1.

Two doses of cyclophosphamide (60 mg/kg) is administered to Patient X on the seventh and sixth day prior to administration of ex vivo expanded NK cells. Five doses of fludaribine (25 mg/m²) is administered to Patient X on each of the five days prior to administration of NK cells. NK cells (2.5×10¹⁰) are subsequently infused over 30 minutes via intravenous administration into the Patient X.

Patient X is subsequently evaluated for reduction in tumor volume.

### EXAMPLE 3

This example demonstrates a clinically-applicable method of preparing NK cells for adoptive transfer into cancer patients in accordance with one embodiment of the invention.

Leukapheresis

Patient peripheral blood lymphocytes (PBLs) are removed by leukapheresis consisting of 7.5 liter exchange lasting about 3 hours for blood sampling. The cells are subsequently purified by centrifugation on a Ficoll cushion.

Lymphocytes are tested by cytolysis assays, cytokine release, limiting dilution analysis, and other experimental studies. Immunological monitoring consists of quantifying NK cells reactivity by using established techniques, such as limiting dilution analysis, in vitro sensitization of bulk cultures, Elispot assays, FOXp3 levels, and levels of CD4⁺/CD25⁺ cells. FOXp3 levels are evaluated by TaqMAN and levels of CD4⁺/CD25⁺ cells by flow cytometry at one month after therapy and is repeated at two months. Immunological assays are standardized by the inclusion of (1) pre-infusion PBMC and (2) an aliquot of the NK cells cryopreserved at the time of infusion. A variety of tests including evaluation of specific lysis and cytokine release, limiting dilution analysis of precursor frequency, ELISA-spot assays, and lymphocyte subset analysis are used to evaluate response to melanoma antigens. In general, differences of 2 to 3 fold in these assays are indicative of true biologic differences. In addition, measurement of CD4⁺/CD8⁺ T cells and CD56⁺/CD3⁻ cells are conducted, including studies of CD4+/CD25+ cells and FOXp3 levels.

Large scale expansion of NK cells from CD3 depleted PBMC for adoptive transfer

The procedure described here is used to expand NK (natural killer) cells isolated from patient PBMCs by CD3 depletion. These cells are used to treat patients with metastatic malignancies after pre-treatment with a non-myeloablative chemotherapy regimen.

The following materials are used in the method of expanding NK cells: Ca²⁺-, Mg²⁺-, Phenol red-free BioWhittaker* Hanks' balanced salt solution (BBSS) ( ); AIM-V medium (GIBCO, Life Technologies; Grand Island, N.Y.); Human serum, type AB (Approved source with appropriate COA); Recombinant human IL-2 (10⁶ CU/ml, Chiron Corp., Emeryville, Calif.)*; Anti-CD3 monoclonal antibody (Orthoclone OKT3®, Ortho Biotech Products; Raritan, N.J.); Gentamicin sulfate, 50 mg/ml, stock (BioWhittaker—Omit if patient is allergic to gentamicin); L-Glutamine, 29.2 mg/ml, stock (Mediatech; Herndon, Va.); Penicillin/Streptomycin (10,000 units Pen/ml, 10,000 μg Strep/ml; BioWhittaker—Omit if patient is allergic to penicillin); Fungizone (Amphotericin B) 250 μg/ml, stock (Bristol-Myers Squibb Co.; Princeton, N.J.—Omit if patient is allergic to Fungizone); Ciprofloxacin, 10 mg/ml stock (Bayer; West Haven, Conn.—Omit if patient is allergic to ciprofloxacin); Albumin (Human) 25%, USP, (Plasbumin-25, Bayer); 0.9% Sodium chloride, USP (Baxter); Nalge filters; 0.8, 0.45, and 0.22 um (1 package of each; Nalge Company, A Subsidiary of Sybron, Rochester, N.Y.); Sterile water for injection, USP (10 ml; American Pharmaceutical Partners, Inc.; Los Angeles, Calif.); Centrifuge tubes, 50 ml and 250 ml; Plastic pipets, sterile 5, 10, 25 and 50 ml; Tissue culture plates, sterile 24; Tissue culture flasks, 175 cm²; Syringes, sterile, 3 ml, 6 ml, and 60 ml; Hypodemic Needles, 19 and 25 gauge; 3-way Stopcock with Luer Lock, sterile (Medex, Dublin, Ohio); Sampling site coupler, (Baxter/Fenwal, Deerfield, Ill.); Solution transfer set, (Baxter/Fenwal, Deerfield, Ill.); Lifecell adapter set, (Baxter/Fenwal, Deerfield, Ill.); Interconnecting jumper tube, 8″ (GIBCO, Life Technologies; Grand Island, N.Y.); Solution transfer pump, (Baxter/Fenwal, Deerfield, Ill.); Culture bags, PL732 1 liter (Nexell Therapeutics, Irvine, Calif.); Culture bags, PL732 3 liter (Nexell Therapeutics, Irvine, Calif.). Note: 1000 Cetus units (CU)=6000 International units (IU); All materials in contact with cells or their media are supplied sterile. Universal Precautions are used when working with human cells, tissues, or blood. All aspirated culture fluids are collected in a Wescodyne-containing trap.

The following procedure is used to expand NK cells:

Cell Culture Media

AIM V medium is used with 25 mM HEPES (pH 7.0), penicillin G (100 U/ml), streptomycin (100 μg/ml), gentamicin (50 μg/ml), beta-mercaptoethanol (5.5×10″5 M), and 10% human serum. The human serum is pre-selected in our laboratory to support NK growth and maintain antitumor activity after expansion.

Preparing Feeder Cells (Autologous PBMC)

Feeder cells are autologous peripheral blood mononuclear cells (PBMC). Each individual leukapheresis must pass sterility tests. The patient is leukapheresed on the day of the CD3 depletion. Once PBMC are received, the cells are divided into two 250 conical tubes are centrifuged at 2000 rpm for 10 minutes in a Sorvall RC3B centrifuge. The supernatant is aspirated and the cells are washed in HBSS, centrifuged again, this time at 800 rpm to deplete platelets. Supernatant is once again removed, the cells resuspended in 200 mLs HBSS and counted. After the cell number is determined, sufficient cells are set-aside for autologous feeders and the remaining portion is subjected to the CD3 depletion procedure. The autologous feeder cells are kept on ice during processing and irradiation to minimize cell clumping. The cells are irradiated with 4,000 cGy, using an MS Nordion Gammacell 1000, Model 38.3 irradiator with a Csl37 source. Clumping, which often occurs in the feeder cells, is thought to be the result of cell lysis and DNA release. The clumps are often not readily dispersed. Clumps should be allowed to settle and their use avoided.

CD3 Depletion Procedure Using the Clinimacs

This protocol describes the clinical scale depletion of CD3⁺ cells labeled with CliniMACS CD3 MicroBeads using the CliniMACSPlus Instrument.

The following materials and equipment are used: Leukapheresis product containing up to 40×10⁹ total cells and up to 15×10⁹ CD3⁺ cells; CliniMACS CD3 MicroBeads, Order No. 176-01; CliniMACSPluls Instrument, Miltenyi Biotec, e.g. Order No. 155-02, software version 2.3×; 1 CliniMACS Tubing Set, Miltenyi Biotec, e.g. Order No. 162-01, 168-01; 1 Pre-System Filter, Miltenyi Biotec, Order No. 181-01; 1 Luer/Spike Interconnector, Miltenyi Biotec, Order No. 187-01; CliniMACS PBS/EDTA buffer, Miltenyi Biotec, e.g. Order No. 705-25; Human Serum Albumin (HSA) or Bovine Serum Albumin (BSA) as supplement to CliniMACS PBS/EDTA buffer, final concentration 0.5%; Transfer Bags 600 ml, Miltenyi Biotec, Order No. 190-01;. Centrifuge, suitable for bag processing; Digital Balance; Sterile Tubing Welder, e.g. Terumo Sterile Connection Device TSCD® SC-201A or 1 Transfer Pack for pooling and/or storage of blood components “Octopus Bag”, Miltenyi Biotec, e.g. Order No. 184-01; Plasma extractor; Orbital Shaker; Sampling Site Coupler; Tubing Slide Clamps or Scissor clamps.

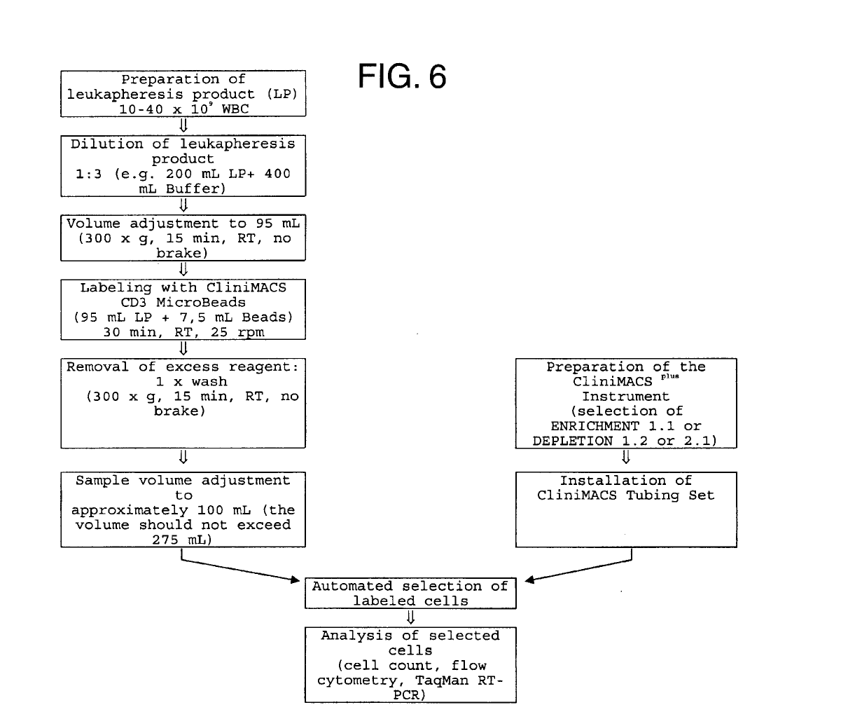

The depletion of CD3 positive cells is performed by immunomagnetic labeling of CD3 expressing cells and enrichment or depletion of these cells from the target fraction by automatic cell separation using the CliniMACS^{plus} Instrument. The enriched labeled CD3⁺ cells or the CD3 depleted fraction of unlabeled target cells is collected in the Cell Collection Bag. The flow chart shown in FIG. 6 gives a step by step overview of a positive selection or depletion using CliniMACS CD3 MicroBeads following an In-Bag-Preparation protocol (normal scale preparation).

Product (Microbead) Specifications

MACS (Iron-dextran) colloid super-paramagnetic Microbeads conjugated to monoclonal mouse anti-human CD3 antibody in PBS buffer stabilized with 0.03% (w/v) Poloxamer 188 (Isotype: Mouse IgG2a Clone: 3G10B1A6). The product is tested for sterility and endotoxins. One vial of CliniMACS® CD25 MicroBeads (7.5 mL) is sufficient for the labeling of CD3 positive cells from up to 40×10⁹ WBC. One vial contains 7.5 mL of CliniMACS CD3 reagent in a sterile nonpyrogenic solution. Each vial contains 7.5 mL of an iron-dextran colloid conjugated to monoclonal mouse anti-human CD3 antibody in PBS buffer stabilized with 0.03% (w/v) Poloxamer 188 (Manufacturer: Miltenyi Biotec GmbH, D-51429 Bergisch Gladbach, Germany; Distributed by Miltenyi Biotec Inc., Auburn Calif. 95603 USA).

Immunomagnetic Labeling

The content of one vial of CliniMACS CD3 MicroBeads is optimized and dosed by Miltenyi Biotec and is sufficient for labeling of up to 15×10⁹ CD3 positive cells out of a total leukocyte number of up to 40×10⁹ cells, the capacity of the system (capacity determined for high expressors after PHA stimulation). Since the number of total cells to be labeled rarely exceeds 10×10⁹ cells, one vial of reagent provides Microbeads in excess of expected yields.

The leukapheresis product is prepared in normal fashion without the ficoll step. The empty Cell Preparation Bag is weighed prior to transferring the leukapheresis product into the Cell Preparation Bag. The volume of the leukapheresis product is determined by weighing the filled Cell Preparation Bag and subtracting the empty bag weight. A small aliquot of the leukapheresis product is used to determine the total number of leukocytes, the percentage of target cells, and the viability. The leukapheresis product is diluted 1:3 (˜200 mL of product up 600 ml) with CliniMACS PBS/EDTA Buffer (supplemented with 0.5% HSA or BSA) and the cells are centrifuged at 300×g for 15 minutes without brake. The amount of buffer to be added is calculated using the following equation: Weight of buffer=Weight of leukapheresis product to be added (g)×2

The cells are spun down at 300×g, 15 min, room temperature at +19° C. to +25° C., without brake. The supernatant is removed and the sample is adjusted to a labeling volume of 95 mL, taking care to not disturb the cell pellet. One vial of CliniMACS CD3 MicroBeads, is added to 10 mL of air and mixed carefully. The cell preparation bag is incubated for 30 minutes at controlled room temperature (+19° C. to +25° C.) on an orbital shaker at 25 rpm. Buffer is added to a final volume of 600 mL for cell washing and the cells are spun down for 15 minutes at room temperature at 300×g without brake. Supernatant is removed as much as possible from the Cell Preparation Bag and the cells are resuspended. The cell concentration is adjusted after the washing step to less than or equal to 0.4×10⁹ total cells/mL. Based upon the recommended cell concentration and capacity of the CD3 depletion (40×10⁹ cells), the final sampling volume of the leukapheresis product for loading on the CliniMACSPlus Instrument does not exceed 100 mL, although the capacity is 275 mL. The labeled leukapheresis product is filtered through a blood filter to remove cell clumps. A 0.5 mL sample is transferred to a sample tube for flow cytometric analysis. The cell concentration, the viability, and the frequency/number of the target cells are determined. The final sampling volume of the leukapheresis product is applied to the CliniMACSPlus Instrument and the depletion 2.1 program is selected for depletion of CD3⁺ cells. Upon completion of the enrichment or depletion program, the enriched labeled CD3⁺ cells or the CD3 depleted fraction of unlabeled target cells is collected in the Cell Collection Bags. Collection bags containing CD3 depleted cell fraction, CD3⁺ cell fraction or waste is weighed and a small volume sample is taken to determine at least the cell concentration, the viability, and the frequency/number of the target cells. Since the CliniMACS Tubing Set and collection bags are disposable units, required cleaning of the device is limited to cleaning with an antiseptic solution, such as Bacillol plus or Meliseptol, at regular intervals or after each application, according to standard protocols for device decontamination.

Automated Separation

The CliniMACS^{plus} Instrument is switched on and select a suitable program is selected according to the chosen separation strategy. For depletion of CD3⁺ cells, DEPLETION 2.1 is recommended. Note that selection program DEPLETION 2.1 is limited to Tubing Sets Order No. 165-01 (or 161-01) and 168-01 (or 162-01). DEPLETION 2.1 is recommended for maximum depletion efficiency. The choice is confirmed by pressing “ENT” and a tubing set is selected. The Order No. of the selected tubing set is entered. Selection program DEPLETION 2.1 is a “staged loading” program. It includes a query for the following parameters to adjust the selection sequence to each individual sample and to provide important information on the required buffer and bag volumes: WBC concentration; percentage of labeled cells; total volume of the sample ready for loading on the CliniMACS Tubing Set. The instructions given on the instrument screen are followed and an appropriate bag is connected to the tubing set using a Luer/Spike Interconnector (Order No. 187-01). The slide clamp of the Luer/Spike Interconnector is ensured that it is open. If more than 1 L of buffer is needed, two buffer bags are connected using a Plasma Transfer Set with two couplers (Order No. 186-01). The second port of one of the buffer bags is used for the connection to the tubing set. The instructions on the instrument screen are followed for the installation of the tubing set and the automated separation program is started. After the separation has been finished, the weight of Cell Collection Bag is determined and a sample is taken for flow cytometry analysis. The weight and cell concentration of the positive fraction, negative fraction and waste bag is determined.

Preparing the Master Mix

A master mix is prepared by combining AIM V supplemented with 10% human AB serum, followed by OKT3, feeder cells (irradiated, autologous PBMC), and finally the responder cells (CD3 depleted fraction) as listed in Table 1. To provide a control culture flask to verify that the feeder cells are irradiated, an appropriate volume of master mix is held without CD3 depleted cells. To generate cells for patient treatment, 1 L bottles are commonly used and 900 mL of master mix per bottle are made. Because 100 mL of Master Mix per 175 cm² flask are used, the data in Table 1 is converted to a multiple of 9 to simplify setting up large numbers of flasks. The following antibiotics are used, depending on the nature of the culture and patient drug allergies: penicillin, streptomycin, gentamicin, amphotericin B, and ciprofloxacin. Test Expansion is used to determine whether the CD3 depleted cells (subsequently NK) are able to expand and maintain antitumor activity in the expansion. Test Expansions differ from Rx Expansions in size (Table 1) and in the procedure for culture expansion. Rx Expansions are expanded into culture bags, as described below. Test expansions are expanded into upright 75 cm² flasks.

TABLE 1

| | 175 cm² flask (Rx | | 25 cm² flask | |
|---|---|---|---|---|
| Component | Expansion) | | (Rest REP) | |
| AIM V +10% human AB | 100 | ml | 10 | ml |
| OKT3 (1.0 mg/ml stock) | 3.0 | μl | 30 | ng/ml |
| IL-2 (6,000,000 IU/ml stock) | 100 | μl | 6000 | IU/ml |

| | | |
|---|---|---|
| Autologous PBMC* | 1 × 10⁸ | 1 × 10⁷ |
| Responder cells | 1 × 10⁷ | 1 × 10⁶ |

*Pretreated with 4,000 cGy irradiation

NK Expansion Cell Culture

100 mL of the master mix are added to each flask. The flasks are incubated upright at 37° C. in 5% CO₂-95% air (day 0). On day 5, after cells have settled by gravity to the bottom of each flask, half of the cell-free medium (˜50 mL) is removed by aspiration and a volume equal to that removed of a fresh mixture of AIM V medium supplemented with 5% Human AB serum, 6,000 IU/ml IL-2, and 250 μl of 5 mg/ml fungizone is added back.

Counts are done starting at Day 7. If the viable cell count is above 0.5×10⁶/ml, an additional 100 mL of AIM V is supplemented with 5% Human AB serum, 6,000 IU/ml IL-2, and 250 μl of 5 mg/ml fungizone. Another count is done at day 10. If the count is above 0.5×10⁶/ml, the cultures are transferred to Baxter 3-liter bags by adding the contents of 3 flasks (200 mL each) to each bag. Also an equal volume (300 mL) of fresh medium consisting of AIM V with penicillin G (100 U/ml), streptomycin (100 μg/ml), L-glutamine (2 mM), Cipro (10 μg/ml), Fungizone (1.25 μg/ml), 6,000 IU/ml IL-2, and 5% human serum is added if needed to bring the cell concentration down to 0.5×10⁶/ml. Bag cultures are split rather than exceeding 1800 mL per bag. If the viable cell count in flasks is too low, the transferring of cultures to bags is delayed. After transferring cells to bags, the viable cell count is monitored every day or two and fresh AIM V with IL-2 (no human serum) is added as needed to keep the cell concentration between about 5×10⁵ and 2×10⁶/ml. Cultures are commonly allowed to reach the higher cell concentrations by the day of the harvest, which commonly is on day 21.

During the rapid expansion of NK for patient treatment, cultures are sampled for quality control tests, including cell viability (frequently during the culture period), antitumor immune activity (as early as day 10), cell-surface phenotypes (after day 10), sterility (including 2-3 days before the harvest and the day of the harvest), and endotoxin levels (the day of the harvest).

### EXAMPLE 4

This example illustrates the adoptive transfer of autologous NK cells into a cancer patient that has undergone lymphodepleting chemotherapy for the treatment of cancer in accordance with the invention.

Patients undergo apheresis and the cells obtained are used for the in vitro generation of autologous natural killer lymphocytes prepared as described in Example 1 or Example 3. Patients then receive the non-myeloablative lymphocyte depleting preparative regimen of cyclophosphamide on days −8 and −7 and fludarabine on days −6 through −2. On day 0, patients will receive the infusion of autologous natural killer lymphocytes and then begin the first cycle of high-dose Aldesleukin. Cells must meet the criteria in the Certificate of Analysis (COA): Infused Cell Product illustrated in Table 2.

TABLE 2

Patient
Date(s) of pheresis for cell product
collection
Date(s) of CD3+ depletion and
cryopreservation of final product
Date of cell infusion

Tests performed on final product

| Test | Method | Limits | Result | Initials/Date |
|---|---|---|---|---|
| Cell Viability¹ | trypan blue exclusion | >70% | | |
| Total Viable cell number¹ | visual microscopic | >10⁹ | | |
| | count | <10¹¹ | | |
| CD56+ CD3− CELLS | FACS Analysis² | >70% | | |
| Lysis assay¹ | CR-51 release² | >20% at 10:1 | | |
| Microbiological studies | gram stain^{1,3} | no micro- | | |
| | | organisms seen | | |
| | aerobic culture^{1,3} | no growth | | |
| | fungal culture^{1,3} | no growth | | |
| | Anaerobic culture^{1,3} | no growth | | |
| | mycoplasma test³ | Negative | | |
| Endotoxin | Limulus assay¹ | 5 E.U./kg | | |

¹Performed on the final product. Results are available at the time of infusion
²Analysis will be performed 3-10 days prior to infusion
³Performed 2-4 days prior to infusion, results are available at the time of infusion but may not be definitive.
⁴Lysis assay uses established cell lines

The Aldesleukin regimen is used in all Surgery Branch protocols (720,000 IU/kg intravenously, every 8 hours for up to 5 days, maximum 15 doses). Inclusion and exclusion criteria set forth in Tables 3 and 4 are followed. About four to six weeks later, patients are evaluated to determine tumor response and toxicity. Immunologic studies are performed including the evaluation of circulating natural killer cells as assessed by the presence of CD56⁺CD3⁻ cells and Foxp3 expression.

TABLE 3
INCLUSION CRITERIA

| | |
|---|---|
| a | Patients must have previously received high dose IL-2 and have |
| | been either non-responders (progressive disease) |
| | or have recurred. |
| b | Patients who are 18 years of age or older, must have |
| | measurable metastatic melanoma or metastatic kidney cancer |
| | and no tumor reactive T cells available for cell transfer therapy. |
| c | Patients of both genders must be willing to practice birth control |
| | for four months after receiving the preparative regimen. |
| d | Clinical performance status of ECOG 0, 1. |
| e | Absolute neutrophil count greater than 1000/mm³. |
| f | Platelet count greater than 100,000/mm³. |
| g | Hemoglobin greater than 8.0 g/dl. |
| h | Serum ALT/AST less than three times the upper limit of normal. |
| i | Serum creatinine less than or equal to 1.6 mg/dl. |
| j | Total bilirubin less than or equal to 2.0 mg/dl, except inpatients |
| | with Gilbert's Syndrome who must have a total bilirubin less |
| | than 3.0 mg/dl. k. Must be willing to sign a durable power of |
| | attorney. |

TABLE 4
EXCLUSION CRITERIA

| | |
|---|---|
| a | Less than four weeks has elapsed since any prior systemic therapy at the time the |
| | patient receives the preparative regimen, or less than six weeks since prior |
| | nitrosurea therapy. |
| b | Women of child-bearing potential who are pregnant or breastfeeding because of |
| | the potentially dangerous effects of the preparative chemotherapy on the |
| | fetus or infant. |
| c | Life expectancy of less than three months. |
| d | Systemic steroid therapy required. |
| e | Any active systemic infections, coagulation disorders or other major medical |
| | illnesses of the cardiovascular, respiratory or immune system, as evidenced |
| | by a positive stress thallium or comparable test, myocardial infarction, cardiac |
| | arrhythmias, obstructive or restrictive pulmonary disease. |
| f | Any form of autoimmune disease (such as autoimmune colitis or Crohn's Disease). |
| g | Seropositive for HIV antibody. (The experimental treatment being evaluated in |
| | this protocol depends on an intact immune system. Patients who are HIV |
| | seropositive can have decreased immune competence and thus be less |
| | responsive to the experimental treatment and more susceptible to its toxicities.) |
| h | Seropositive for hepatitis B or C antigen |
| i | Seronegative for Epstein-Barr virus (EBV). |
| j | Patients who are not eligible to receive high-dose Aldesleukin as evaluated by |
| | the following: |
| | Patients who are 50 years old or greater who do not have a normal stress cardiac |
| | test (stress thallium, stress MUGA, dobutamine echocardiogram, or other |
| | stress test) will be excluded. |
| | Patients who have history of EKG abnormalities, symptoms of cardiac ischemia |
| | or arrhythmias who do not have a normal stress cardiac test (stress thallium, |
| | stress MUGA, dobutamine echocardiogram, or other stress test) will be excluded. |
| | Patients with a prolonged history of cigarette smoking or symptoms of |
| | respiratory dysfunction who do not have a normal pulmonary function test as |
| | evidenced by a FEV_{L} <60% predicted will be excluded. |
| | Patients who experienced toxicities during prior IL-2 administration that would |
| | preclude redosing with IL-2, i.e. myocardial infarction, mental status changes |
| | requiring intubation, bowel perforation or renal failure requiring dialysis. |

A. Drug Administration

The drug/cell administration regimen is performed according to Table 5.

TABLE 5
CVCLOPHOSPHAMIDE AND FLUDARABINE
Day −8 and −7

| | |
|---|---|
| 1 am | Hydrate: 0.9% Sodium Chloride 2.6 ml/kg/hr 10 meq/1 KCL (starting 11 hours |
| | pre-cyclophosphamide and continue hydration until 24 hours after last |
| | cyclophosphamide infusion) |
| 11 am | Ondansetron (approximately 0.15 mg/kg/dose [depending upon pharmacy |
| | guidelines]) IV q 8 hours × 2-4 days) may be given for nausea. |
| | Furosemide 10-20 mg iv. |
| 12 pm | Cyclophosphamide 60 mg/kg/day × 2 days IV in 250 ml D5W with Mesna 15 mg/kg/ |
| (NOON) | day × 2 days over 1 hr. Maximum dose not to exceed doses calculated |
| | on body weights greater than 140% of the maximum ideal body weight |
| | (Metropolitan Life Insurance Company). |
| 1 pm | Begin to monitor potassium level every 12 hours until hydration is stopped. |
| | KCl will be adjusted to maintain serum potassium levels in the normal range. |
| 1 pm | Begin mesna infusion at 3 mg/kg/hour intravenously diluted in a suitable |
| | diluent (see pharmaceutical section) over 23 hours after each |
| | cyclophosphamide dose. Maximum mesna doses not to exceed doses calculated |
| | on body weights greater than 140% of the maximum ideal body weight |
| | (Metropolitan Life Insurance Company). |
| Day −6 | Stop IV hydration (24 hours after last cyclophosphamide dose) If urine output |
| | <1.5 ml/kg/hr give additional 20 mg furosemide iv. If body weight >2 kg over |
| | pre cyclophosphamide value give additional furosemide 10-20 mg iv. |
| Day −6 to | Fludarabine 25 mg/m²/day IVPB daily over 15-30 minutes for 5 days. |
| Day −2: | Maximum dose not to exceed doses calculated on body weights greater than |
| | 140% of the maximum ideal body weight (Appendix 1: Metropolitan Life |
| | Insurance Company). |
| Day −1 | No drug administration on this day. |
| Day 0 | Autologous NK cells will be infused intravenously over 20-30 minutes and |
| | filgrastim will be started at 10 mcg/kg/day daily subcutaneously until |
| | neutrophil count >0.5 × 10⁹/l. Administration of Aldesleukin will be initiated at |
| | 720,000 IU/kg IV every 8 hours for up to 5 days (maximum 15 doses). |

Prior to the beginning of chemotherapy, patients undergo a 20 to 30 liter apheresis in the Surgery Branch apheresis unit while enrolled on 03-C-0277 (Cell Harvest and Preparation for Surgery Branch Adoptive Cell Therapy Protocols) to obtain a target number of greater than 10¹⁰ PBMC. The preparation of the natural killer cells is as detailed in Example 1 or 3. Cells are infused intravenously on day 0 (two days after the last dose of fludarabine) in the Patient Care Unit over 20 to 30 minutes.

The following measures can be taken towards infection prophylaxis:

Pneumocvstis Carini Pneumonia

All patients receive the fixed combination of trimethoprim and sulfamethoxazole [SMX] as double strength (DS) tab (DS tabs=TMP 160 mg/tab, and SMX 800 mg/tab) P.O. bid twice weekly, beginning on day −8 and continue prophylaxis for at least 6 months post chemotherapy and until the CD4 count is above 200 on two consecutive follow up lab studies. The required dose is TMP/SMX-DS, 1 tablet PO bid twice a week on Tuesday and Friday.

Patients with sulfa allergies receive aerosolized Pentamidine 300 mg per nebulizer within one week prior to admission and continue monthly until the CD4 count is above 200 on two consecutive follow up lab studies and for at least 6 months post chemotherapy.

Herpes Virus Prophylaxis

Patients with positive HSV serology are given acyclovir starting 24 hours after the last dose of Fludarabine (day −1), orally at a dose of 800 mg twice a day which is continued until absolute neutrophil count is greater than 1000/ml. Reversible renal insufficiency has been reported with IV but not oral acyclovir. Neurologic toxicity including delirium, tremors, coma, acute psychiatric disturbances, and abnormal EEGs have been reported with higher doses of acyclovir. Should this occur, a dosage adjustment is made or the drug is discontinued. Acyclovir is not used concomitantly with other nucleoside analogs which interfere with DNA synthesis, e.g. ganciclovir. In renal disease, the dose is adjusted as per product labeling.

Fungal Prophylaxis (Fluconazole)

Patients start Fluconazole 400 mg p.o. 24 hours after the last dose of Fludarabine (day −1) and continue until the absolute neutrophil count is greater than 1000/mm³.

CMV disease sometimes occurs in profoundly immunocompromised patients like the ones who receive treatment under this protocol. CMV is monitored monthly by PCR during the first three months after the procedure (the blood can be shipped to the NIH for testing). Active CMV disease is treated as per standard of care with antivirals (ganciclovir or foscamet), plus or minus IVIG. Asymptomatic CMV reactivation is monitored without intervention. Persistently rising levels of CMV DMA in the blood is treated pre-emptively after consultation with the Infectious Diseases Consult Service of the NIH.

Empiric Antibiotics

Patients start on broad spectrum antibiotics, either a 3^{rd} or 4^{th} generation cephalosporin, a quinolone, or a carbapenem at single fever greater than or equal to 38.3° C. once or two temperatures of 38.0° C. or above at least one hour apart simultaneously with an ANC less than 500/mm³. Aminoglycosides are avoided unless clear evidence of sepsis. Infectious disease consultation is obtained from all patients with unexplained fever or any infectious complications.

Blood Product Support

Using daily CBC's as a guide, the patient receives platelets and packed red blood cells (PRBC's) as needed. Attempts are made to keep Hb >8.0 gm/dl, and pits 20,000. All allogeneic blood products are irradiated. Leukocyte filters are utilized for all blood and platelet transfusions to decrease sensitization to transfused WBC's and decrease the risk of CMV infection.

Aldesleukin (IL-2) Administration

Aldesleukin is administered at a dose of 720,000 lU/kg as an intravenous bolus over a 15 minute period every eight hours beginning on the day of cell infusion and continuing for up to 5 days.

The aldesleukin regimen is delayed for at least 6 hours after cell infusion in the first 3 patients in order to clearly differentiate potential cell administration toxicities from the toxicities observed with high dose aldesleukin infusion. If no excessive (>grade 3) or unanticipated cell infusion toxicities are observed, the FDA is notified and aldesleukin therapy is initiated after the cell infusion in subsequent patients.

Doses are skipped depending on patient tolerance. Doses are skipped if patients reach Grade III or IV toxicity due to Aldesleukin except for the reversible Grade III toxicities common to Aldesleukin such as diarrhea, nausea, vomiting, hypotension, skin changes, anorexia, mucositis, dysphagia, or constitutional symptoms and laboratory changes as detailed in Appendix 6 and 7. If this toxicity is easily reversed by supportive measures then additional doses are given.

Tables 6 to 8 demonstrate the percentage and total number of circulating NK cells in three patients who are treated.

TABLE 6

| Patient EB | Day 9 | Day 10 | Day 12 | Day 15 | Day 53 | Day 80 |
|---|---|---|---|---|---|---|
| % NK Cell | 92.0 | 90.0 | 90.0 | 90.0 | 69.0 | 56.0 |
| NK Cell No. | 1437 | 4618 | 7901 | 5154 | 975 | 465 |

TABLE 7

| Patient AB | Day 5 | Day 7 | Day 9 | Day 12 | Day 48 |
|---|---|---|---|---|---|
| % NK Cell | 93.0 | 95.0 | 91.0 | 87.0 | 55.0 |
| NK Cell No. | 318 | 1339 | 1105 | | 708 |

TABLE 8

| Patient JW | Day 5 | Day 7 | Day 13 | Day 19 | Day 24 | Day 46 |
|---|---|---|---|---|---|---|
| % NK Cell | 88.0 | 86.0 | 65.0 | 79.0 | 69.0 | 39.0 |
| NK Cell No. | 1352 | 1608 | 1251 | 1040 | 1840 | 358 |

The foregoing illustrates the adoptive transfer of autologous NK cells into a cancer patient that has undergone lymphodepleting chemotherapy for the treatment of cancer in accordance with an embodiment of the invention.

### EXAMPLE 5

This example demonstrates in vitro stimulation of NK cells with interleukin-12 (IL-12).

NK cells were prepared and cultured in vitro as generally described in Example 1. Varying amounts of IL-12 (0, 3, or 30 ng/mL) were added to the culture, and IL-12 was allowed to stimulate the NK cells for 1 day. After 1 day, the NK cells were evaluated for their ability to secrete interferon gamma (IFNγ) and lyse tumor cell targets (melanoma cell line 888 mel). NK cells grown in the presence or absence of IL-12 efficiently lysed tumor cell targets. However, only the NK cells grown in the presence of IL-12 secreted IFNγ in response to tumor cells (see Table 9).

TABLE 9

| | IL-12 (ng/mL) | | | IL-12 (ng/mL) | | |
|---|---|---|---|---|---|---|
| | (IFNγ pg/mL) | | | (% lysis at 10:1) | | |
| Patient | 0 | 3 | 30 | 0 | 3 | 30 |
| 1 | 52 | 876 | >1000 | 51 | 55 | 76 |
| 2 | 2 | 387 | 402 | 17 | 26 | 37 |
| 3 | 3 | 271 | 267 | 28 | 36 | 42 |
| 4 | 88 | >1000 | >1000 | 69 | 67 | 89 |
| 5 | 14 | 579 | 558 | 64 | 64 | 75 |

The foregoing illustrates that stimulation of autologous NK cells with IL-12 results in secretion of IFNγ in the presence of tumor cells.

### EXAMPLE 6

This example demonstrates lysis of tumor cells exposed to activated autologous NK cells in combination with tumor-specific monoclonal antibodies.

PBMCs were collected from a patient 10 days after receiving adoptively transferred NK cells in combination with lymphodepleting chemotherapy. At this time point, the PBMC population consisted of 90% NK cells. PBMCs were cryopreserved and later thawed and cultured in vitro in the presence or absence of IL-2 for two days. The ability of these NK cells to lyse tumor cells was then evaluated after coating the tumor cells with a monoclonal antibody. Specifically, lysis of two HER2/neu-positive cell lines (SKOv3 and SKBr₃) and one HER2/neu-negative cell line (MDA MB 468) was evaluated after incubation with the anti-HER2/neu monoclonal antibody Herceptin® (Genentech, South San Francisco, Calif.), or a control monoclonal antibody directed against CD20 (Rituxan®, Genentech, South San Francisco, Calif.).

NK cells cultured in the absence of IL-2 did not lyse tumor cell targets coated with the control antibody, but exhibited considerable lysis of HER2/neu-positive cell lines coated with the anti-HER2/neu antibody. These results suggest that coupling the adoptive transfer of activated autologous NK cells with monoclonal antibody therapy may enhance cancer treatment.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating cancer in a host that has undergone lymphodepleting chemotherapy, the method comprising administering to the host a composition comprising ex vivo-activated autologous natural killer (NK) cells in an amount effective to treat cancer.

2. The method of claim 1, further comprising administering IL-2 to the host.

3. The method of claim 1, wherein the ex vivo-activated autologous natural killer (NK) cells are prepared by ex vivo co-culturing the NK cells with irradiated peripheral blood mononuclear cells (PBMCs) that are autologous to the NK cells.

4. The method of claim 3, wherein the cells are co-cultured in the presence of Interleukin-2 (IL-2) and OKT3.

5. The method of claim 3, wherein the cells are co-cultured in the presence of Interleukin-12 (IL-12).

6. The method of claim 1, wherein the ex vivo-activated autologous natural killer cells comprise a heterologous nucleic acid sequence encoding a cytokine or an Fc receptor.

7. The method of claim 6, wherein the cytokine is IL-12.

8. The method of claim 6, wherein the Fc receptor is CD64.

9. The method of claim 1, wherein the host has undergone a nonmyeloablative lymphodepleting chemotherapy.

10. The method of claim 1, wherein the host has undergone adoptive transfer of autologous tumor infiltrating lymphocytes (TIL).

11. The method of claim 1, further comprising administering to the host a tumor-specific monoclonal antibody.

12. The method of claim 1, wherein the host is a mammal.

13. The method of claim 12, wherein the mammal is a human.

14. The method of claim 1, wherein the cancer is melanoma, renal cell carcinoma, or breast, prostate, or colon cancer.

15. The method of claim 1, wherein cells of the cancer do not express any Major Histocompatibility Complex (MHC) Class I molecules.

16. The method of claim 1, wherein cells of the cancer express an MHC molecule.

17. A method of preparing a composition comprising NK cells, the method comprising
(i) depleting CD3⁺ cells from a population of PBMCs comprising NK cells to provide a CD3⁺ cell-depleted PBMC population, wherein the CD3⁺ cell-depleted PBMC population comprises NK cells,
(ii) co-culturing cells from the CD3⁺ cell-depleted PBMC population with irradiated PBMCs, wherein the irradiated PBMCs are autologous to the NK cells.

18. The method of claim 17, wherein the population of PBMCs from which CD3⁺ cells are depleted is obtained by leukapheresis of a blood sample of a host.

19. The method of claim 17, comprising
(i) obtaining a population of PBMCs by leukapheresis of a host,
(ii) depleting CD3⁺ cells from a first portion of the population of PBMCs, thereby obtaining a CD3⁺ cell-depleted PBMC population, and irradiating a second portion of the population of PBMCs, thereby obtaining irradiated PBMCs, and
(iii) co-culturing the CD3⁺ cell-depleted PBMC population with the irradiated PBMCs.

20. The method of claim 17, wherein the host is a mammal.

21. The method of claim 20, wherein the mammal is a human.

22. The method of claim 17, wherein only CD3⁺ cells are depleted from the population of PBMCs comprising NK cells prior to co-culturing the CD3⁺ cell-depleted population with irradiated PBMCs.

23. The method of claim 17, wherein the cells are co-cultured in the presence of IL-2 and OKT3.

24. The method of claim 17, wherein the cells are co-cultured in the presence of IL-12

25. The method of claim 17, further comprising introducing into the NK cells a heterologous nucleic acid sequence encoding a cytokine or an Fc receptor

26. The method of claim 24, further comprising introducing into the NK cells a heterologous nucleic acid sequence encoding a cytokine or an Fc receptor.

27. The method of claim 26, wherein the cytokine is IL-12.

28. The method of claim 26, wherein the Fc receptor is CD64.

29. The method of claim 17, wherein the NK cells of the prepared composition are able to lyse cancer cells.

30. The method of claim 29, wherein the cancer cells are melanoma cells.

31. A composition prepared by the method of claim 17.

32. A composition prepared by the method of claim 24.

33. A composition prepared by the method of claim 25.

34. A method of treating or preventing a disease or an immunodeficiency in a host, comprising administering to the host a composition of claim 31 in an amount effective to treat the disease or immunodeficiency.

35. A method of treating or preventing a disease or an immunodeficiency in a host, comprising administering to the host a composition of claim 32 in an amount effective to treat the disease or immunodeficiency.

36. A method of treating or preventing a disease or an immunodeficiency in a host, comprising administering to the host a composition of claim 33 in an amount effective to treat the disease or immunodeficiency.

37. A method of treating or preventing a disease or an immunodeficiency in a host, wherein the method comprises administering to the host a composition comprising autologous natural killer (NK) cells in an amount effective to treat the disease or the immunodeficiency, wherein the autologous NK cells are ex vivo-activated by co-culturing with irradiated autologous PBMCs.

38. The method of claim 37, wherein the immunodeficiency is AIDS.

39. The method of claim 37, wherein the disease is an autoimmune disease or a cancer.

40. The method of claim 39, wherein the cancer is melanoma, renal cell carcinoma, or breast, prostate, or colon cancer.
